(19) United States
(12) Patent Application Publication
(10) Pub. No.: US 2026/0083894 A1
(43) Pub. Date: Mar. 26, 2026

(54) POSTOPERATIVE CHEST DRAINAGE DEVICE

(71) Applicant: REDAX S.P.A., Poggio Rusco (IT)

(72) Inventor: Lucio GIBERTONI, Mirandola (IT)

(21) Appl. No.: 19/107,732

(22) PCT Filed: Jun. 8, 2023
(86) PCT No.: PCT/EP2023/065406

§ 371 Date: Feb. 28, 2025

(30) Foreign Application Priority Data
Sep. 1, 2022 (IT) 102022000017949

(51) Int. Cl. A61M 1/00; A61M 39/00; A61M 39/24

(52) U.S. Cl. CPC A61M 1/69; A61M 39/24; A61M 2039/0009; A61M 2202/0014; A61M 2202/02; A61M 2202/04; A61M 2205/3331; A61M 2205/7536; A61M 2209/088; A61M 2210/10

(57) ABSTRACT

A postoperative chest drainage device for draining fluids that originate from the chest of a patient and which has liquids and/or gases, the device includes: a bag for collecting the liquids and an input connector adapted to be connected to a drainage duct for the passage of the fluids that originate from the chest toward the bag, and an air-liquid separation device which is arranged outside of the bag, between the input connector and the bag, and is configured to allow the passage of the gases, when they are present in such fluids, toward the environment outside the bag and to direct the liquids, when present in the fluids, toward the bag

### CROSS-REFERENCE TO RELATED APPLICATIONS

This application is a 35 U.S.C. § 371 National Stage patent application of PCT/EP2023/065406, filed on 8 Jun. 2023, which claims the benefit of Italian patent application 102022000017949, filed on 1 Sep. 2022, the disclosures of which are incorporated herein by reference in their entirety.

### TECHNICAL FIELD

The present disclosure relates to postoperative chest drainage device for treatment in hospital and at home of patients who, generally following chest surgery, necessitate draining of fluids from the chest.

### BACKGROUND

Nowadays, the reduction in invasiveness of surgical procedures and the application of protocols for reducing postoperative treatment times (e.g. ERAS—Enhanced Recovery After Surgery), are increasing the demand for medical devices that allow the continuation of postoperative therapies at home, i.e. outside hospital facilities. This kind of treatment brings with it undeniable advantages such as: greater availability of hospital beds for treating a greater number of patients; reduction in the overall healthcare costs for a given procedure; reduction in episodes of hospital-originated complications (not least, recently, the contraction of postoperative infections and/or of viral diseases like SARS-COV-2); improvement of the overall quality of life of the patient who can spend the postoperative period at home.

Since most medical devices for postoperative chest drainage have been designed for hospital use, these are not suitable for use at home because they require precautions and a level of training that are not compatible with the average patient.

Finally, designing a medical device for home use must allow the patient to be able to go about their daily activities as naturally as possible and in the greatest safety, and therefore the devices must be completely closed-circuit with respect to the outside environment and must have extremely compact dimensions.

Currently, there are hospitals that authorize the fast-track discharge of a patient, which consists of allowing the post-operative patient to return home to spend part of the postoperative period outside the hospital, in so doing freeing up the bed for the next patient.

However, the state of the art of the chest drainage devices available is not entirely satisfactory for authorizing a fast-track discharge, in that it is composed of medical devices that are designed either to obtain the best quality of post-operative care, or to aim for miniaturization at the cost of some capabilities. As an illustrative categorization of conventional chest drainage devices, nowadays there are: standard chest draining devices, chest draining devices with autonomous vacuum units, Heimlich valves with a bag, and devices for treating pneumothorax.

Standard chest drainage devices are containers with a typical capacity of 2000 ml and these are generally provided with numerous one-way valves for adjustment. They are designed to be placed beside the bed of the patient and are often connected to the centralized hospital suction network. These standard chest drainage devices can be moved around, but only within the hospital environment, because the dimensions do not allow easy transfers. A patient would never be discharged with such devices.

Chest drainage devices with autonomous vacuum units are similar to the previous devices, but are provided with suction units powered by rechargeable batteries and they have software for managing and controlling features. Typically they have a collection capacity of 300-2000 ml and they can be more compact than standard chest drainage devices, but they are still cumbersome owing to the presence of the suction unit and the container. The presence of the vacuum unit makes it possible to maintain suction during transport and, in some cases, it provides clinical information on the postoperative period. Their operation requires specialist hospital staff and therefore they are not suitable for home use.

The Heimlich valve with a bag is a device made up of a one-way valve connected, at one end, to the drainage tube and, at the other end, to a collection bag. Although compact and straightforward, this device is used very rarely for the fast-track discharge of a patient because it is rather cumbersome and difficult to fasten (and it can be used only for the least serious cases). It is not at all easy for the patient to go about their daily activities, and there is a high risk of involuntary tugging which can lead to dislocation of the catheter. Furthermore, the simultaneous presence of air and liquids in the fluid drained from the patient generally entails a very complex handling of the collection bag, which can often be swollen because the air enters the bag together with the liquid, with consequent interruption of the draining.

Devices for treating a pneumothorax are rather simple and compact devices and are characterized by the presence of a one-way valve that allows the outflow of the air originating from the chest of the patient, toward the outside environment. They have very small dimensions and therefore they afford freedom of movement to the patient. Against this, since they are designed to treat pneumothorax, they are adapted only to drain air, while for any liquids that may be simultaneously present they have an extremely small collection capacity (less than 50 ml). This limitation makes them unsuitable for the fast-track discharge of a patient after surgery, since generally a portion of liquids and a portion of air are always present simultaneously, and both must be effectively drained.

Some of these known chest drainage devices are equipped with an expulsion valve for the drained air, which is located in a rigid container for collecting liquid which is fastened to the body of the patient; this solution has the disadvantage that, if the patient lies down on their back or side, or if it is operated incorrectly, the drained liquid can exit from the air expulsion valve.

### SUMMARY

The aim of the present disclosure is to provide a postoperative chest drainage device that is capable of solving the above-mentioned problems and overcoming the above-mentioned limitations of the background art.

Within this aim, the present disclosure provides a chest drainage device that is adapted for home use.

The disclosure also provides a chest drainage device that does not require a high level of specialization for operation.

The disclosure further provides a chest drainage device that allows the expulsion of the gaseous fraction of the drained fluids safely and continuously, and which prevents the problem of swelling of the bag owing to this gaseous fraction.

Another feature of the disclosure is providing a chest drainage device that has a simple and compact structure.

Another feature of the disclosure is providing a chest drainage device that is very safe and reliable and is also versatile.

This aim and these and other advantages which will become more apparent hereinafter are achieved by providing a postoperative chest drainage device according to the independent claim.

### BRIEF DESCRIPTION OF THE DRAWINGS

Further characteristics and advantages of the disclosure will become better apparent from the detailed description of a preferred, but not exclusive, embodiment of a postoperative chest drainage device, illustrated by way of non-limiting example with the aid of the accompanying drawings wherein:

FIG. 1 is a front elevation view, from the front, of a possible embodiment of the chest drainage device according to the disclosure;

FIG. 2 is a rear view of the chest drainage device of FIG. 1;

FIG. 3 is a perspective view of the air-liquid separation device comprised in the chest drainage device;

FIG. 4 is an exploded view of some components of the air-liquid separation device; and

FIG. 5 is an exploded view of some more components of the air-liquid separation device.

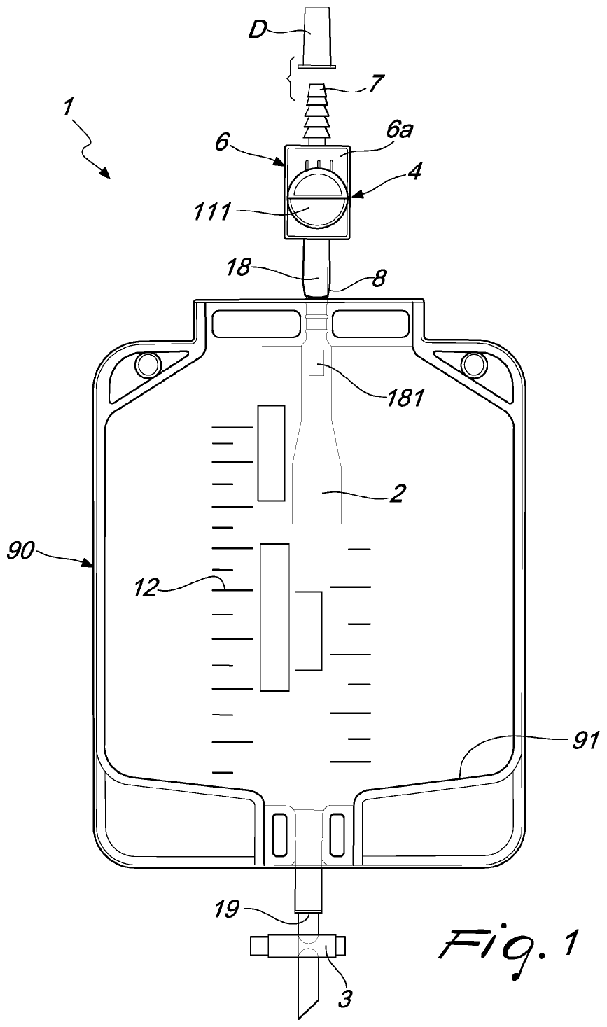

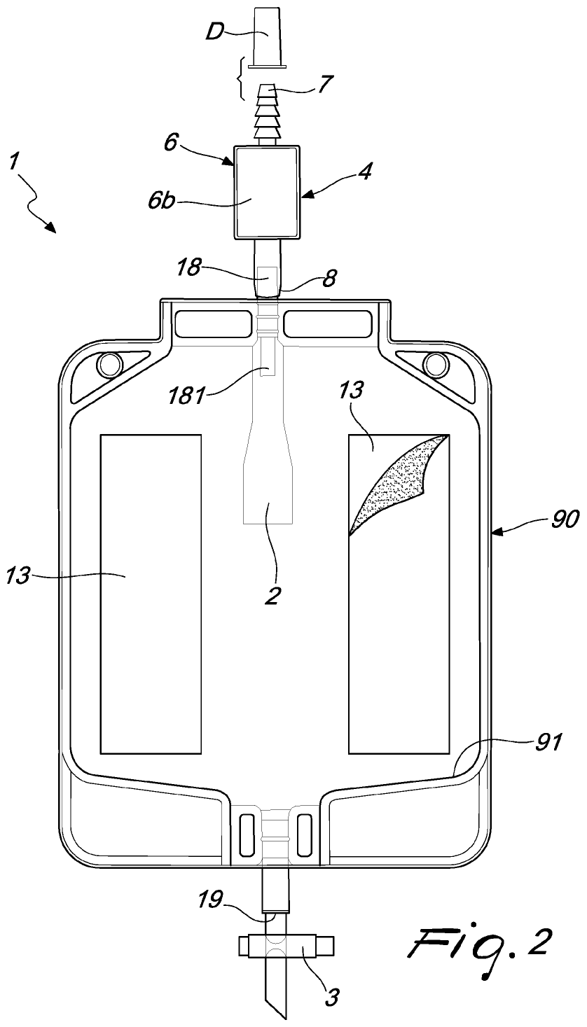

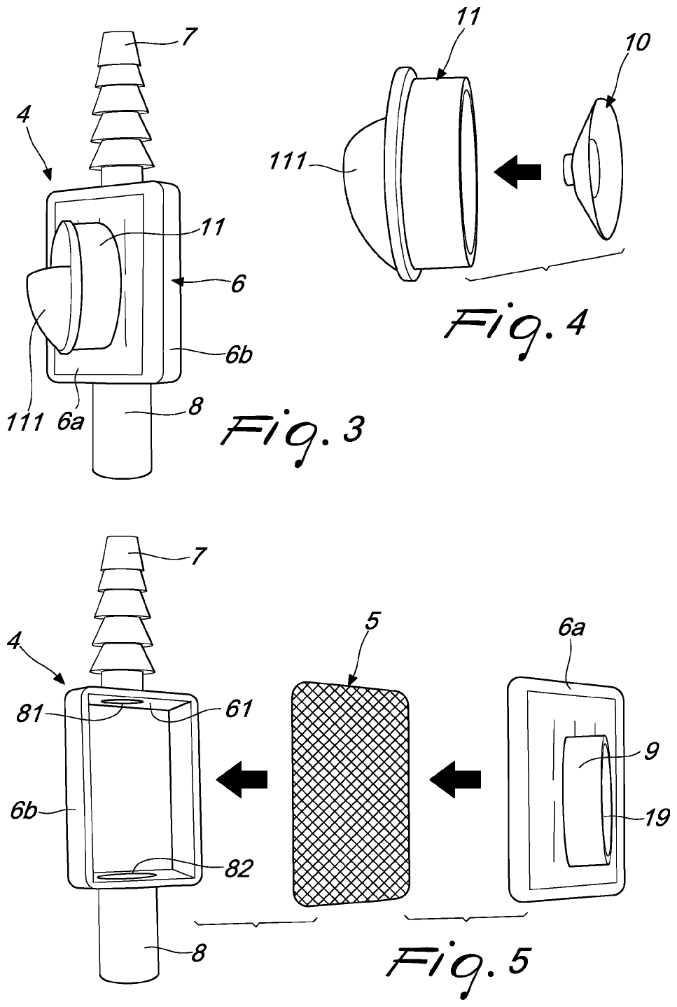

### DETAILED DESCRIPTION OF THE DRAWINGS

With reference to the figures, the postoperative chest drainage device, generally designated by the reference numeral 1, is used for draining fluids that originate (are drained) from the chest of a patient, and which can comprise liquids and/or gases.

The drainage device 1 comprises a bag 90 for collecting the liquids, which can be provided in a known manner, and which, obviously, defines internally a collection chamber for liquids 91.

The bag 90 is provided with a duct or entry opening 181 for the liquids to enter the bag 90 (into the collection chamber for liquids 91) and preferably also with an exit port 19 for the liquids in the bag 90 to exit.

The drainage device 1 also comprises an input connector 7 which is adapted to be connected to a drainage duct D (for example to the outlet of a catheter or drainage tube or of any other device adapted to place the bag 90 in fluid connection with the interior of the chest) for the passage of fluids originating from the chest toward the bag 90; the input connector 7 is therefore in fluid communication with the bag 90.

Advantageously, the input connector 7 is tapered and even more preferably it has a “fish bone” shape, so that it can be connected to tubes of different diameters.

According to the disclosure, the drainage device 1 comprises an air-liquid separation device 4 which is located outside of the bag 90, between the input connector 7 and the bag 90.

This air-liquid separation device 4 is configured to allow the passage of gases, when they are present in the drained fluids, toward the environment outside the bag 90 (i.e. so as to direct them outside the bag 90).

The air-liquid separation device 4 is configured to also direct the liquids, when they are present in the drained fluids, toward the bag 90.

In this manner, the bag 90 is filled only by the liquid component of the drained fluids, while the gaseous component is continuously vented outside, without entering the bag 90.

Preferably, the collection bag of the liquids 90 has a capacity of 300-500 ml.

In the preferred embodiments, the drainage device 1 comprises a one-way valve 2, preferably of the flutter valve type, which is included in the bag 90 and located at the point of entry to the bag 90 where the liquids enter (i.e. coupled to the duct or entry opening 181) and is configured to prevent the exit of the liquids from the duct or entry opening 181 toward the air-liquid separation device 4 and therefore toward the patient: the function of this one-way valve 2 is therefore to prevent, under any circumstances (e.g. accidental squeezing of the bag by the patient), the reflux of liquids toward the patient.

Optionally, the exit port 19 is coupled to a lower faucet 3 for the periodic emptying of the bag 90, which allows the liquids to exit from the bag so as to restore the collection capacity without needing to replace the device.

In the preferred embodiments, which include the illustrated embodiment, the amount of liquid collected is quantifiable by virtue of the presence of a special graduated scale 12 on the front face of the bag 90.

Optionally, the chest drainage device 1 comprises one or more adhesive bands 13 for fastening the bag 90 to the body of the patient. The adhesive bands 13 are arranged on the rear face of the bag 90 and enable a fastening that is solid and safe, but comfortable, so that the patient is capable of easily going about their daily activities without the typical bulk of the devices currently present on the market, by ensuring the correct distance between the outlet of the drainage catheter and the bag for collecting the liquids.

Turning now to the preferred embodiment of the air-liquid separation device 4, this comprises a container body 6, preferably made of plastic, which defines a fluid passage chamber 61 inside it.

The container body 6 comprises: a fluid entry opening 81 for the entry of the fluids, which arrive from the input connector 7, into the fluid passage chamber 61; a liquid exit opening 82 for the exit of the liquids from the fluid passage chamber 61 in the direction of the bag 90; a gas exit opening 19 for the exit of the gases from the fluid passage chamber 61 toward the outside environment.

A hydrophobic filtering membrane 5 is contained inside the container body 6 and separates the fluid passage chamber 61 from the gas exit opening 19 (and therefore separates the fluid entry opening 81 and the liquid exit opening 82 from the gas exit opening 19).

The hydrophobic filtering membrane 5 is configured to allow the passage through it of gases (present in the fluids passing through the fluid passage chamber 61) and at the same time to prevent the passage of liquids. This is achieved, for example, with a membrane 5 that has a suitable porosity, such as to allow the passage of gases, and which has been objected to a hydrophobic treatment on the surface directed toward the fluid passage chamber 61.

Conveniently, the fluid passage chamber 61 is substantially parallelepiped and the hydrophobic filtering membrane 5 is substantially rectangular, so as to have a sufficiently ample surface to facilitate the passage of gases.

Preferably, the input connector 7 is fixed directly to the container body 6 so that the outlet of the input connector 7 corresponds to the fluid entry opening 81. In the preferred and illustrated embodiment, the input connector 7 and the container body 6 are part of a single body.

Preferably, the container body 6 is fixed directly to the fluid collection bag 90, by way of an outlet connector 8 (which for example is coupled to an inlet neck 18 of the bag 90), so that the liquids exiting from the liquid exit opening 82 flow into the bag 90 through the outlet connector 8 and therefore through the entry opening or duct 181 of the bag 90. Alternatively, the container body 6 can be connected to the bag 90 by way of a flexible tube or other duct.

In more detail, the fluid entry opening 81 and the liquid exit opening 82 are arranged at two opposite ends of the fluid passage chamber 61; therefore the container body 6 is connected at a first (upper) end to the input connector 7 and at a second (lower) end to the bag 90. The gas exit opening 19 is positioned on a side face of the container body 6.

Preferably, the fluid entry opening 81 and the liquid exit opening 82 have mutually parallel or coinciding axes (coinciding with an outflow axis of the fluids), while the gas exit opening 19 has an axis which is perpendicular, or at least transverse, with respect to the axes of the other two openings.

In the specific embodiment illustrated, the container body 6 comprises two parts 6a, 6b: a first, main part 6b which defines the fluid passage chamber 61 on five sides, and a closing part 6a which acts as a cap, closing the fluid passage chamber 61 on the last side, and on which the gas passage opening 19 is present. The hydrophobic filtering membrane 5 is positioned adjacent to the closing part 6a.

Preferably, the air-liquid separation device 4 comprises a one-way valve 10 which is configured to allow the passage of gases only in output and not in input, so as to prevent the entry of air toward the patient when the patient is breathing in. Conveniently, such one-way valve 10 is associated with the gas passage opening 19 and is configured to allow the passage of gases only in output from the gas passage opening 19 and not in input; even more preferably the air-liquid separation device 4 comprises a seat 9 which is adapted to accommodate such one-way valve 10, such seat 9 being for example formed by a wall that surrounds the gas passage opening 19.

Preferably, the one-way valve 10 is of the “umbrella” type, even more preferably made of soft silicone.

Note that the one-way valve 10 is positioned on the opposite side of the gas passage opening 19 with respect to the hydrophobic filtering membrane 5: the former is positioned outside the container body 6, the latter inside.

Optionally, the one-way valve 10 is covered by a cap 11, for example also fitted on the seat 9, which at least contributes to keeping the one-way valve 10 in the seat, in addition to protecting it.

According to an optional and advantageous characteristic, the cap 11 is provided with a pocket 111, preferably rounded in shape, which defines inside it a cavity that is in fluid connection with the gas passage opening 19. This is adapted to contain a liquid (for example physiological saline) in order to enable the detection of the outflow of gas: in fact, the outflow of air from the valve 10 is “invisible” to the naked eye and therefore a medical operator would find it impossible to determine the presence, if any, of a persistent air leak, while the presence of a small quantity of liquid (even just a few droplets) in the cavity through which the air passes, at the outflow point of the air, makes it possible to see such a leak in the form of bubbling, which will be more intense the greater the leak.

It is also possible to have the pocket 111 that defines a cavity inside it in fluid connection with the gas passage opening 19, in a different manner, independently of the cap 11 and of the specific position of the one-way valve 10, still with the purpose of being filled with liquid for detecting the outflow of gas.

The operation of the chest drainage device, although clear and evident from the foregoing description, is summed up below with reference to the illustrated preferred embodiment.

The drainage device 1 is connected to a drainage catheter D by means of the input connector 7.

The fluid drained from the patient and conveyed by the catheter enters the connector 7, arriving in the air-liquid separation device 4 (entering the fluid passage chamber 61 inside the container body 6). Here the gaseous fraction of the drained fluid passes easily through the hydrophobic filtering membrane 5 by virtue of the wide surface and the porosity of the membrane 5, which offers low resistance to the passage of air.

After passing through the hydrophobic filtering membrane 5, the air can easily exit from the air-liquid separation device 4 through the one-way valve 10; this, in addition to permitting the outflow of air without offering resistance, is capable of preventing the reentry of air toward the patient when the patient is breathing in, which creates negative pressures with respect to the outside environment.

The presence of an air expulsion valve (consisting of the one-way valve 10), “upstream” of the collection bag 90, makes it possible to prevent air from flowing into the bag 90, causing it to swell.

Air leaks of the patient can be detected in the form of bubbling when the cavity present in the pocket 111 is filled with a few droplets of physiological saline.

The liquid fraction of the fluid drained from the patient is prevented from passing through the membrane 5 by virtue of the hydrophobic treatment of the surface thereof that faces toward the entry of the fluid. Therefore, the liquid is separated from the gaseous fraction and proceeds through the outlet connector 8 toward the collection bag 90.

At the inlet of the bag 90 the one-way valve 2 is provided, which prevents the reflux of the liquid toward the patient. The amount of liquid collected is quantifiable using the graduated scale 12.

It is possible to periodically empty the collected drained liquid using the faucet 3.

During use, the bag 90 can be fastened to the patient using the adhesive bands 13 which are present on the rear side of the bag 90.

It is important to note that, by virtue of the presence of the two one-way/non-return valves 10, 2, the drainage device 1 is completely closed-circuit with respect to the outside environment, to the advantage of safety, in particular in the home use situation where the patient cannot be considered an expert user.

In particular, with respect to standard and/or digital chest drainage devices, the drainage device 1 according to the disclosure has a very simplified structure and therefore is suitable for hospital use but, especially, for home use where it would not be possible to use other devices since they require a high level of specialization for operation.

With respect to conventional products of the “Heimlich valve with a bag” type, the chest drainage device 1, according to the disclosure, makes it possible to separate the air from the liquid before entering the bag 90; therefore, the expulsion of the air fraction from the drained fluid can occur easily and safely and continuously. In a Heimlich valve with a bag, in fact, the presence of air would cause the collection bag to swell, with the need for frequent manual interventions to empty it. In the device according to the disclosure, by contrast, the filtering membrane 5 and the one-way valve 10 are configured to offer minimal resistance to the passage of air, thus preventing problems such as hypertensive pneumothorax or subcutaneous emphysema.

In the drainage device 1, according to the disclosure, the presence of the hydrophobic membrane 5 prevents the drawback of the drained liquid flowing out from the air expulsion valve, in any situation, while still allowing the correct removal of the drained air.

In practice it has been found that the chest drainage device according to the present disclosure achieves the intended aim and objects, in that it is particularly adapted for home use and does not require a high level of specialization for operation.

Another advantage of the chest drainage device according to the disclosure consists in that it allows the expulsion of the gaseous fraction of the drained fluids safely and continuously, while also preventing the problem of swelling of the bag owing to this gaseous fraction.

Further advantages of the chest drainage device according to the disclosure consist in having a structure that is simple and compact and in that it is very safe and reliable and is also versatile.

The chest drainage device thus conceived is susceptible of numerous modifications and variations all of which are within the scope of the appended claims.

Moreover, all the details may be substituted by other, technically equivalent elements.

In practice the materials employed, and the contingent dimensions and shapes, may be any according to requirements and to the state of the art.

1-11. (canceled)

12. A postoperative chest drainage device for draining fluids that originate from the chest of a patient and which comprise liquids and/or gases, comprising:
a bag for collecting the liquids;
an input connector, adapted to be connected to a drainage duct for the passage of said fluids that originate from the chest toward the bag;
and further comprising an air-liquid separation device arranged outside of said bag, between the input connector and the bag, and configured to:
allow the passage of the gases, when the gases are present in said fluids, toward the environment outside the bag, and
direct the liquids, when present in said fluids, toward the bag.

13. The drainage device according to claim 12, wherein the air-liquid separation device comprises a container body which defines a fluid passage chamber inside;
said container body comprising:
a fluid entry opening for entry of the fluids, which arrive from the input connector, into the fluid passage chamber;
a liquid exit opening for the exit of the liquids from the fluid passage chamber in the direction of the bag; and
a gas exit opening for exit of the gases from the fluid passage chamber toward the outside environment.

14. The drainage device according to claim 13, wherein a hydrophobic filtering membrane is enclosed inside the container body and separates the fluid passage chamber from the gas exit opening and is configured to allow the passage of gases through the membrane and to prevent the passage of liquids.

15. The drainage device according to claim 13, wherein the fluid entry opening and the liquid exit opening have mutually parallel or coinciding axes; the gas exit opening having an axis which is perpendicular, or transverse, with respect to said axes of the fluid entry opening and of the liquid exit opening.

16. The drainage device according to claim 12, wherein the air-liquid separation device comprises a one-way valve which is configured to allow the passage of gases only in output, so as to prevent the entry of air toward the patient.

17. The drainage device according to claim 16, wherein the one-way valve) is associated with the gas passage opening and is configured to allow the passage of the gases only in output from the gas passage opening.

18. The drainage device according to claim 16, further comprising a pocket which defines a cavity inside which is in fluid connection with the gas passage opening and is adapted to contain a liquid in order to allow the detection of the outflow of gases.

19. The drainage device according to claim 18, wherein the one-way valve is arranged outside the container body and is covered by a cap which at least contributes to keep said one-way valve in a seat thereof; said cap being provided with said pocket.

20. The drainage device according to claim 12, further comprising a one-way valve which is included in the bag and is configured to prevent outflow of the liquids from the bag toward the air-liquid separation device.

21. The drainage device according to claim 12, wherein said bag comprises an exit port to which a lower faucet for emptying the bag is coupled.

22. The drainage device according to claim 12, further comprising one or more adhesive bands for fastening the bag to the body of the patient.
